(19) United States
(12) Patent Application Publication
(10) Pub. No.: US 2023/0310265 A1
(43) Pub. Date: Oct. 5, 2023

(54) Multi-adjustable clamping-type ultrasonic drug application and massage device

(71) Applicant: Zhijun PENG, Foshan (CN)

(72) Inventor: Zhijun PENG, Foshan (CN)

(21) Appl. No.: 18/093,332

(22) Filed: Jan. 5, 2023

(30) Foreign Application Priority Data
Apr. 1, 2022 (CN) 202210338778.8

(51) Int. Cl. A61H 23/02

(52) U.S. Cl. CPC A61H 23/0245; A61H 2201/0157; A61H 2201/0192

(57) ABSTRACT

A multi-adjustable clamping-type ultrasonic drug application and massage device, including a fixed arm, a sliding arm, a fixed horizontal rod, ultrasonic drug application and massage heads, and a sliding horizontal rod; the fixed arm is fixed at an end of the fixed horizontal rod; the sliding horizontal rod slidably sleeves the fixed horizontal rod; a locking structure is provided between the sliding horizontal rod and the fixed horizontal rod; the sliding arm slidably sleeves the sliding horizontal rod; a locking mechanism is provided between the sliding arm and the sliding horizontal rod; each of the fixed arm and the sliding arm has a fixed supporting arm, a sliding sleeve arm, and a locking nut; the ultrasonic drug application and massage heads are installed on the fixed arm and the sliding arm respectively; the two ultrasonic drug application and massage heads face towards each other, forming a massage space in-between.

### FIELD OF THE INVENTION

The invention relates to the technical field of ultrasonic massage device, in particular to an ultrasonic drug application and massage device.

### BACKGROUND OF THE INVENTION

The existing clamping or wearing-type massage healthcare devices are either clamped or worn on human body neck, waist, leg, or arm, and carry out massage by applying high-frequency vibration to these body parts. In practical application, it is found that two problems are still to be solved. They are as follows:

First, the clamping function of the message device only relies on the elastic nature of the frame materials of the device, for example, the elasticity of the steel or plastic bars. The device is unable to clamp tightly and easily falls off when wearing. During massage by high-frequency vibration, the device may keep detaching and falling off due to loose clamping. This affects the massage effect of the device and also the user experience.

Secondly, the size of the frame of the device is fixed and non-adjustable. Users cannot adjust the device to an optimized wearing position to suit their respective body shapes. An uncomfortable user experience greatly lowers the intention of choosing and using the product and thus affects the sales.

In view of the disadvantages in the prior art, there is still room for improvement for the existing massage device to better fit the actual needs of the users.

### BRIEF SUMMARY OF THE INVENTION

To solve the aforementioned problems, this invention provides a multi-adjustable clamping-type ultrasonic drug application and massage device, where a clamping space of which is adjustable according to users’ needs and the device is simple in structure, easy to use, small in size, safe, secured and comfortable to wear, and provides high-quality massage.

This invention adopts the following technical solution:

A multi-adjustable clamping-type ultrasonic drug application and massage device, comprising a fixed arm, a sliding arm, a fixed horizontal rod, a pair of ultrasonic drug application and massage heads, and a sliding horizontal rod; the fixed arm is fixedly installed at an end of the fixed horizontal rod; the sliding horizontal rod slidably sleeves the fixed horizontal rod; a locking structure is provided between the sliding horizontal rod and the fixed horizontal rod to lock a position of the sliding horizontal rod relative to the fixed horizontal rod after the sliding horizontal rod slides with respect to the fixed horizontal rod; the sliding arm slidably sleeves the sliding horizontal rod; a locking mechanism is provided between the sliding arm and the sliding horizontal rod such that an adjusted position of the sliding arm sliding relative to the sliding horizontal rod is fixed by the locking mechanism; each of the fixed arm and the sliding arm comprises a fixed supporting arm, a sliding sleeve arm, and a locking nut; one of the ultrasonic drug application and massage heads is installed on an end of the sliding sleeve arm of the fixed arm, and another one of the ultrasonic drug application and massage heads is installed on an end of the sliding sleeve arm of the sliding arm; the ultrasonic drug application and massage head on the fixed arm and the ultrasonic drug application and massage head on the sliding arm are arranged to face towards each other, forming a massage space in-between.

Further, the locking structure comprises a pin, pin holes, a return spring and a mounting seat.

Further, the locking mechanism comprises a sliding slot, a locking nut, and a locking block which comprises a locking bolt.

Further, a first horizontal adjustment mechanism is provided on the end of the sliding sleeve arm of the fixed arm, and a second horizontal adjustment mechanism is provided on the end of the sliding sleeve arm of the sliding arm; each of the first horizontal adjustment mechanism and the second horizontal adjustment mechanism is installed with a corresponding ultrasonic drug application and massage head.

The benefits of this invention are: The sliding horizontal rod and the fixed horizontal rod form a horizontal relative position adjustment structure; the fixed arm and the sliding arm form another horizontal relative position adjustment structure; and the fixed supporting arm and the sliding sleeve arm of each of the fixed arm and the sliding arm form a vertical position adjustment structure; when using the device of the present invention, the clamping space may be adjusted easily according to the body shape via the aforementioned adjustment structures, so that the device is comfortable for clamping and wearing and will not fall off easily. Due to the adjustment structures, one single product of this invention can already provide massage to various body parts and satisfy different massaging needs of the users. The adjustment procedures are easy and convenient to users. Furthermore, the adjustment structures allow this invention to fold into smaller size when it is sold in package or not in use, and hence save space for storage and costs for packaging and storage, and is also easier to be transported and carried. The adjustment structures are all simple mechanical structures which do not involve use of electricity, and hence are simple to use, reliable, durable with a low production cost.

### BRIEF DESCRIPTION OF THE DRAWINGS

FIG. 1 is a schematic view of this invention.

FIG. 2 is a first structural view of the main body of this invention.

FIG. 3 is a second structural view of the main body of this invention.

FIG. 4 is an exploded view of the dissembled main body of this invention.

FIG. 5 is a cross-sectional view of the locking structure of this invention.

FIG. 6 is a structural view of the pin of this invention.

FIG. 7 is a cross-sectional view of the ultrasonic drug application and massage head of this invention.

### DETAILED DESCRIPTION OF THE INVENTION

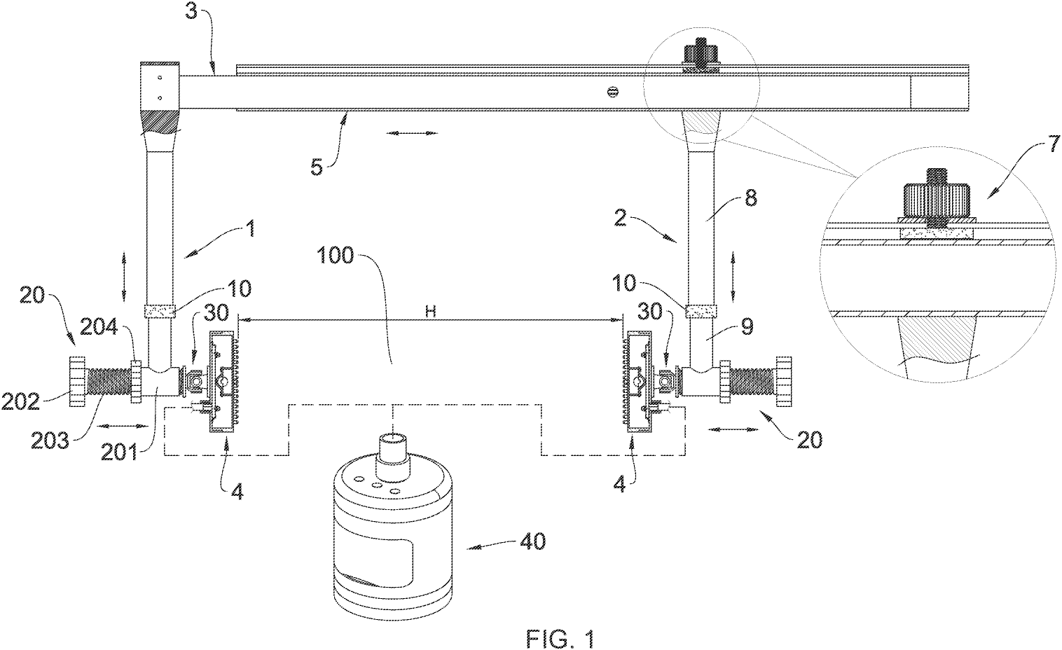

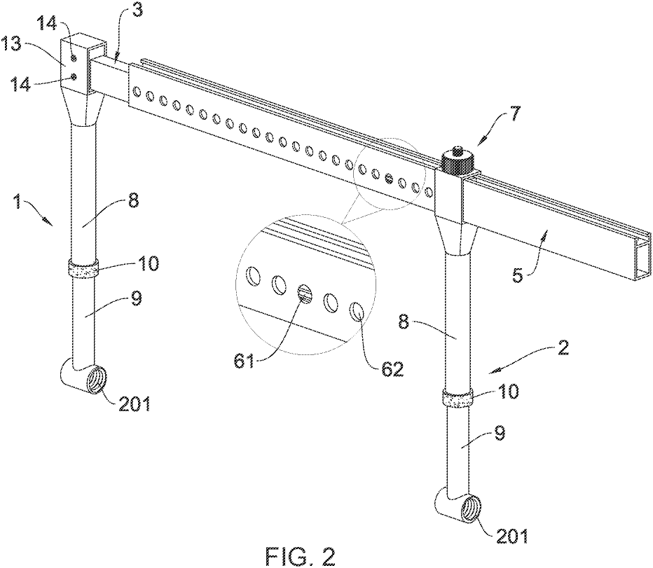

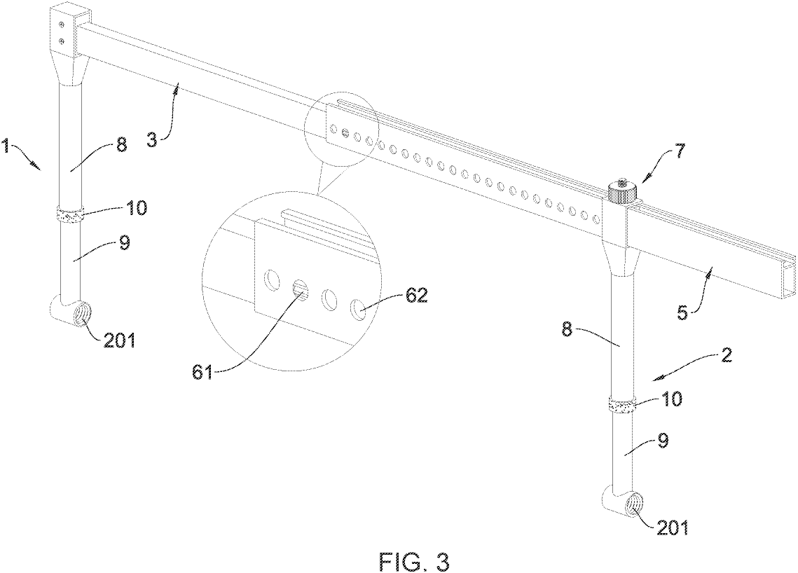

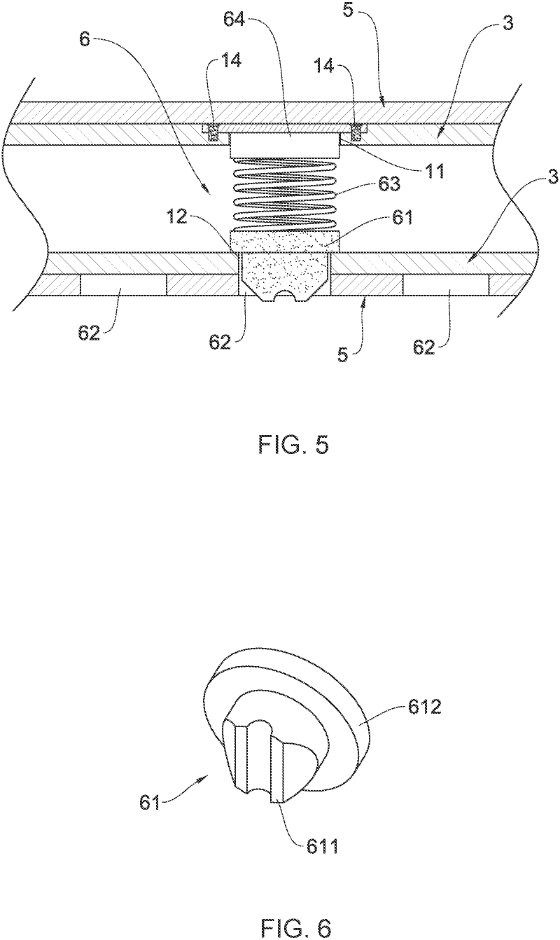

As shown in FIG. 1, a multi-adjustable clamping-type ultrasonic drug application and massage device, mainly comprising a fixed arm 1, a sliding arm 2, a fixed horizontal rod 3, a pair of ultrasonic drug application and massage heads 4 and a sliding horizontal rod 5. The fixed arm 1 is fixedly installed at an end of the fixed horizontal rod 3; specifically, as shown in FIG. 2 to FIG. 4, a top end of the fixed arm 1 is provided with a sleeve joint 13 such that the fixed arm 1 sleeves the end of the fixed horizontal rod 3 via the sleeve joint 13, and screws 14 are provided to pass through the sleeve joint and the fixed horizontal rod so that the sleeve joint and the fixed horizontal rod are fixed together by the screws. The sliding horizontal rod 5 slidably sleeves the fixed horizontal rod 3 such that the sliding horizontal rod 5 may slide on the fixed horizontal rod 3 along a length of the fixed horizontal rod 3 to increase or decrease an overall length defined by the fixed horizontal rod 3 and the sliding horizontal rod 5. A locking structure 6 is provided between the sliding horizontal rod 5 and the fixed horizontal rod 3 to lock a position of the sliding horizontal rod 5 relative to the fixed horizontal rod 3 after overall length adjustment. As shown in FIG. 3 to FIG. 5, the locking structure 6 comprises a pin 61, pin holes 62, a return spring 63 and a mounting seat 64. The pin holes 62 are arranged in a row on a lateral side of the sliding horizontal rod 5 along a length of the sliding horizontal rod 5. The fixed horizontal rod 3 is provided with an installation hole 11 on one lateral side thereof, and a pin mounting hole 12 in communication with the installation hole 11 on an opposite lateral side thereof; the mounting seat 64 is fixed on the installation hole 11 by countersunk head screws 14. The pin 61 is sleeved in the pin mounting hole 12, and the return spring 63 is provided between the mounting seat 64 and the pin 61. When the sliding horizontal rod 5 slides to a position where the pin 61 and one of the pin holes 62 align with each other, the pin 61 is inserted into said one of the pin holes 62 under the resetting power of the return spring 63, thereby fixing a position of the sliding horizontal rod 5 relative to the fixed horizontal rod 3 after the overall length adjustment. As shown in FIG. 6, the pin 61 comprises a pin end 611 and a position limiting ring 612; the pin end 611 allows the pin 61 to disengage from said one of the pin holes 62 when the pin 61 is halfway withdrawn from said one of the pin holes 62; the position limiting ring 612 prevents the pin 61 from being completely pushed out from said one of the pin holes 62. When adjusting, press the pin 61 by hand or by tool, such that the return spring 63 is compressed and the pin 61 is halfway withdrawn from said one of the pin holes 62; push or pull the sliding horizontal rod 5 such that the overall length defined by the sliding horizontal rod 5 and the fixed horizontal rod 3 may be adjusted to longer by pulling the sliding horizontal rod 5 away from the fixed horizontal rod 3 or shorter by pushing the sliding horizontal rod 5 towards the fixed horizontal rod 3.

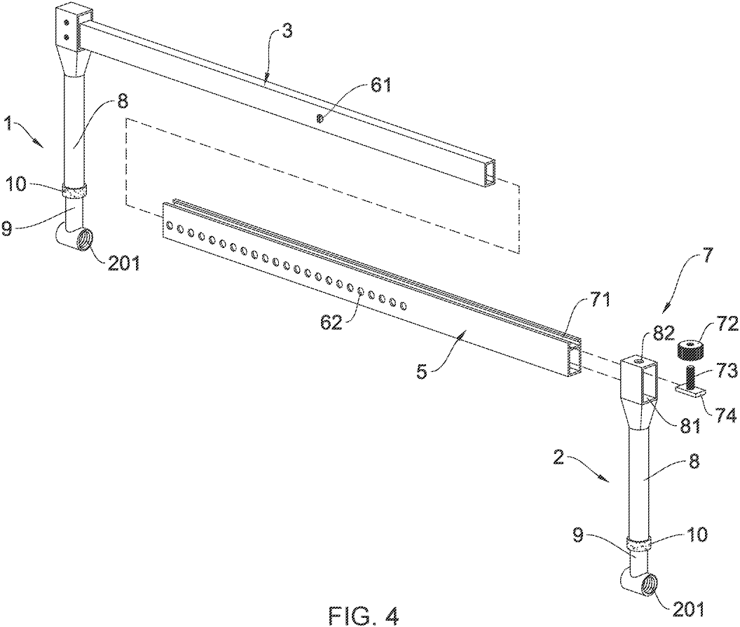

As shown in FIG. 2 and FIG. 3, the sliding arm 2 slidably sleeves the sliding horizontal rod 5; a locking mechanism 7 is provided between the sliding arm 2 and the sliding horizontal rod 5 such that an adjusted position of the sliding arm 2 sliding relative to the sliding horizontal rod 5 may be fixed by the locking mechanism 7. In order to achieve a simpler structure, higher reliability and lower production cost, as shown in FIG. 4, the locking mechanism 7 comprises a sliding slot 71, a locking nut 72, and a locking block 74 which comprises a locking bolt 73; the sliding slot 71 is integrally formed on a top surface of the sliding horizontal rod 5 along the length of the sliding horizontal rod 5. A sleeve component 81 is provided on a top end of a fixed supporting arm 8 of the sliding arm 2, and a through hole 82 is provided on a top side of the sleeve component 81; the locking block 74 is provided in the sleeve component 81; the locking bolt 73 passes through the through hole 82; after the sleeve component 81 slidably sleeves the sliding horizontal rod 5, the locking block 74 is inserted in the sliding slot 71, and the locking nut 72 is screwed on the locking bolt 73.

As shown in FIG. 1 to FIG. 4, the fixed arm 1 and the sliding arm 2 each comprises a fixed supporting arm 8, a sliding sleeve arm 9 and a locking nut 10. The fixed supporting arm 8 is partially provided with a hollow portion in which the sliding sleeve arm 9 is being sleeved into, and the locking nut 10 is provided on an outer end of the hollow portion of the fixed supporting arm 8. When adjusting a length of the fixed arm 1 and the sliding arm 2, rotate the locking nut 10 to an unlocked state, pull the sliding sleeve arm 9 to a required length and then screw the locking nut 10 to a locked state.

As shown in FIG. 1, one ultrasonic drug application and massage head 4 is installed on an end of the sliding sleeve arm 9 of the fixed arm 1, and another ultrasonic drug application and massage head 4 is installed on an end of the sliding sleeve arm 9 of the sliding arm 2; the ultrasonic drug application and massage head 4 on the fixed arm 1 and the ultrasonic drug application and massage head 4 on the sliding arm 2 are arranged to face towards each other, forming a massage space 100 in-between. When using, a body part is placed in the massage space 100 and are clamped and massaged by the pair of ultrasonic drug application and massage heads 4. A length and width of the clamping space 100 may be adjusted by length and/or position adjustments as described above, such that the pair of ultrasonic drug application and massage heads 4 are suitable for clamping and fitting different body parts, for example, arm or waist. Then, the ultrasonic drug application and massage heads 4 are activated to perform massaging by ultrasonic vibration, and mist containing drugs is also transported to the ultrasonic drug application and massage heads 4 via an externally connected drug-supplying device 40, such that drugs are applied on skin during massage.

To horizontally adjust the ultrasonic drug application and massage heads 4, as shown in FIG. 1, a horizontal adjustment mechanism 20 is provided on an end of the sliding sleeve arm 9 of the fixed arm 1 and another horizontal adjustment mechanism 20 is provided on an end of the sliding sleeve arm 9 of the sliding arm 2; each ultrasonic drug application and massage head 4 is installed on a corresponding horizontal adjustment mechanism 20. The horizontal adjustment mechanisms 20 allow users to adjust a clamping position H, which is also a length of the massage space 100; thereby users may adjust the clamping force and be more comfortable during wearing by fine tuning the length of the massage space. Furthermore, in order that the horizontal adjusting mechanisms 20 are simple in structure, easy to adjust, and easy to use with a low production cost, as shown in FIG. 1, each horizontal adjustment mechanism 20 comprises a screw hole seat 201 provided at the end of the corresponding sliding sleeve arm 9, a screw 203 with a handle 202, and a fixing screw nut 204; the screw 203 is installed in the screw hole seat 201, the fixing screw nut 204 is installed on the screw 203, and a corresponding ultrasonic drug application and massage head 4 is installed on a front end of each screw 203. Meanwhile, to obtain a fit and comfortable clamping angle of the ultrasonic drug application and massage heads 4, as shown in FIG. 1 and FIG. 4, a universal joint 30 is provided between the front end of each screw 203 and the corresponding ultrasonic drug application and massage head 4. The universal joint 30 may be a cross and roller type or a ball and trunnion type universal joint.

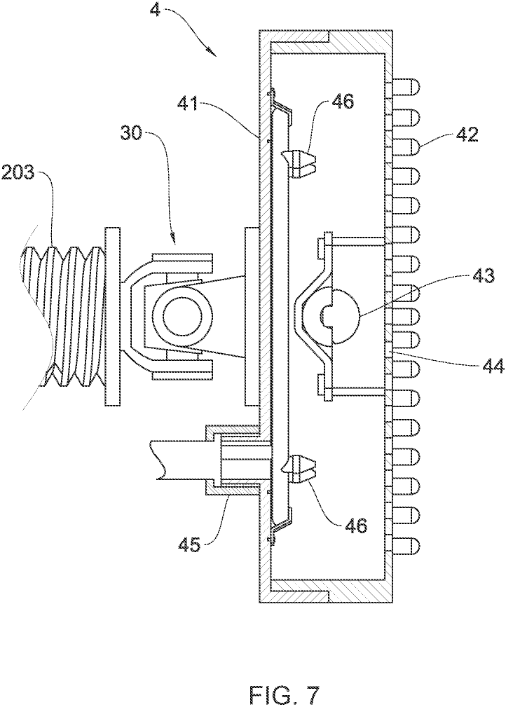

In order that the ultrasonic drug application and massage heads 4 are simple in structure, easy to realize, reliable and durable, as shown in FIG. 7, each of the ultrasonic drug application and massage heads 4 comprises a case 41, a plurality of massage bumps 42, an ultrasonic vibrating component 43, a plurality of drug spray holes 44, and a pipe port 45; the ultrasonic vibrating component 43 is installed in the case 41; the plurality of massage bumps 42 are provided at a front end surface of the case 41; the drug spray holes 44 are provided at spaces between the two massage bumps 42, the pipe port 45 is provided on a back surface or a side surface of the case 41. Besides, to achieve a better ultrasonic energy efficiency ratio, the ultrasonic vibrating component 43 is an ultrasonic transducer of 1 MHz or above, or an ultrasonic vibrating motor of 10,000 RPM or above.

Besides, a circuit board with a control chip may be provided in each ultrasonic drug application and massage head 4 or the drug-supplying device 40 to control the operation of the ultrasonic drug application and massage heads 4 and the drug-supplying device 40; a Bluetooth® module and a WiFi communication module etc. may also be provided on the circuit board and cooperate with a corresponding control APP software, such that the operation of this invention may be controlled by smartphones or tablets. The circuit board is not shown in the drawings and is not described in detail as it is an essential operating component of general electric appliances and belongs to a publicly known technique familiar to those skilled in the art.

The ultrasonic drug application and massage heads 4 perform high-frequency ultrasonic vibration massage and sprays drugs to body parts. When using, connect the drug-supplying device 40 to the ultrasonic drug application and massage head 4s such that high-frequency ultrasonic vibration massage and drug spraying is performed at the same time; the high-frequency ultrasonic message speeds up the flow of blood in the body parts, such that the internal temperature of the muscles increases and the pores on the muscles open to receive the drug molecules, therefore, the drug enters and acts in human body more rapidly and efficiently, thereby enhancing pharmaceutical effects of the drugs and enhancing the general healthcare performance of the device of the present invention.

What is claimed is:

1. A multi-adjustable clamping-type ultrasonic drug application and massage device, comprising a fixed arm (1), a sliding arm (2), a fixed horizontal rod (3), a pair of ultrasonic drug application and massage heads (4), and a sliding horizontal rod (5); the fixed arm (1) is fixedly installed at an end of the fixed horizontal rod (3); the sliding horizontal rod (5) slidably sleeves the fixed horizontal rod (3); a locking structure (6) is provided between the sliding horizontal rod (5) and the fixed horizontal rod (3) to lock a position of the sliding horizontal rod (5) relative to the fixed horizontal rod (3) after the sliding horizontal rod (5) slides with respect to the fixed horizontal rod (3);
the sliding arm (2) slidably sleeves the sliding horizontal rod (5); a locking mechanism (7) is provided between the sliding arm (2) and the sliding horizontal rod (5) such that an adjusted position of the sliding arm (2) sliding relative to the sliding horizontal rod (5) is fixed by the locking mechanism (7);
each of the fixed arm (1) and the sliding arm (2) comprises a fixed supporting arm (8), a sliding sleeve arm (9), and a locking nut (10);
one of the ultrasonic drug application and massage heads (4) is installed on an end of the sliding sleeve arm (9) of the fixed arm (1), and another one of the ultrasonic drug application and massage heads (4) is installed on an end of the sliding sleeve arm (9) of the sliding arm (2); the ultrasonic drug application and massage head (4) on the fixed arm (1) and the ultrasonic drug application and massage head (4) on the sliding arm (2) are arranged to face towards each other, forming a massage space (100) in-between.

2. The multi-adjustable clamping-type ultrasonic drug application and massage device of claim 1, wherein the locking structure (6) comprises a pin (61), pin holes (62), a return spring (63) and a mounting seat (64).

3. The multi-adjustable clamping-type ultrasonic drug application and massage device of claim 2, wherein the pin holes (62) are arranged in a row on a lateral side of the sliding horizontal rod (5) along a length of the sliding horizontal rod (5).

4. The multi-adjustable clamping-type ultrasonic drug application and massage device of claim 2, wherein the fixed horizontal rod (3) is provided with an installation hole (11) on one lateral side thereof, and a pin mounting hole (12) in communication with the installation hole (11) on an opposite lateral side thereof; the mounting seat (64) is fixed on the installation hole (11); the pin (61) is sleeved in the pin mounting hole (12), and the return spring (63) is provided between the mounting seat (64) and the pin (61).

5. The multi-adjustable clamping-type ultrasonic drug application and massage device of claim 1, wherein the locking mechanism (7) comprises a sliding slot (71), a locking nut (72), and a locking block (74) which comprises a locking bolt (73).

6. The multi-adjustable clamping-type ultrasonic drug application and massage device of claim 5, wherein the sliding slot (71) is integrally formed on a top surface of the sliding horizontal rod (5) along a length of the sliding horizontal rod (5); a sleeve component (81) is provided on a top end of the fixed supporting arm (8) of the sliding arm (2), and a through hole (82) is provided on a top side of the sleeve component (81); the locking block (74) is provided in the sleeve component (81); the locking bolt (73) passes through the through hole (82); after the sleeve component (81) slidably sleeves the sliding horizontal rod (5), the locking block (74) is inserted in the sliding slot (71), and the locking nut (72) is screwed on the locking bolt (73).

7. The multi-adjustable clamping-type ultrasonic drug application and massage device of claim 1, wherein a first horizontal adjustment mechanism is provided on the end of the sliding sleeve arm (9) of the fixed arm (1), and a second horizontal adjustment mechanism is provided on the end of the sliding sleeve arm (9) of the sliding arm (2); each of the first horizontal adjustment mechanism and the second horizontal adjustment mechanism is installed with a corresponding ultrasonic drug application and massage head (4).

8. The multi-adjustable clamping-type ultrasonic drug application and massage device of claim 7, wherein each of the first horizontal adjustment mechanism and the second horizontal adjustment mechanism comprises a screw hole seat (201) provided at the end of the corresponding sliding sleeve arm (9), a screw (203) with a handle (202), and a fixing screw nut (204); the screw (203) is installed in the screw hole seat (201), the fixing screw nut (204) is installed on the screw (203), and a corresponding ultrasonic drug application and massage head (4) is installed on a front end of each screw (203).

9. The multi-adjustable clamping-type ultrasonic drug application and massage device of claim 8, wherein a universal joint (30) is provided between the front end of each screw (203) and the corresponding ultrasonic drug application and massage head (4).

10. The multi-adjustable clamping-type ultrasonic drug application and massage device of claim 7, wherein each of the ultrasonic drug application and massage heads (4) comprises a case (41), a plurality of massage bumps (42), an ultrasonic vibrating component (43), a plurality of drug spray holes (44), and a pipe port (45); the ultrasonic vibrating component (43) is installed in the case (41); the plurality of massage bumps (42) are provided at a front end surface of the case (41); the drug spray holes (44) are provided at spaces between the two massage bumps (42), the pipe port (45) is provided on a back surface or a side surface of the case (41); the ultrasonic vibrating component (43) is an ultrasonic transducer of 1 MHz or above, or an ultrasonic vibrating motor of 10,000 RPM or above.
